(19) United States
(12) Patent Application Publication
(10) Pub. No.: US 2013/0211307 A1
(43) Pub. Date: Aug. 15, 2013

(54) NEGATIVE PRESSURE WOUND THERAPY PRODUCT AND METHOD

(71) Applicant: BSN Medical, Inc., (US)

(72) Inventors: John C. Evans, Lancashire (GB); Patrick Schuetz, Hamburg (DE)

(73) Assignee: BSN Medical, Inc., Charlotte, NC (US)

(21) Appl. No.: 13/766,282

(22) Filed: Feb. 13, 2013

Related U.S. Application Data
(60) Provisional application No. 61/597,888, filed on Feb. 13, 2012.

(51) Int. Cl. A61F 13/00; D04B 15/00

(52) U.S. Cl. CPC A61F 13/00068; D04B 15/00
USPC 602/44; 66/231

(57) ABSTRACT

A negative pressure wound therapy bandage including a three-dimensional knitted fabric formed of fibers that are moisture resistant and that define an exudates transport layer for transporting wound exudates away from a wound. The bandage may further include a non-adherent material adapted for preventing the exudates transport layer from adhering to the wound during therapy.

### CROSS-REFERENCE TO RELATED APPLICATION

This application claims priority to U.S. Application No. 61/597,888 filed Feb. 13, 2012, the contents of which are incorporated by reference herein.

### TECHNICAL FIELD AND BACKGROUND OF THE INVENTION

The present invention relates generally to the field of wound care, and more particularly, to a product and method for carrying out negative pressure wound therapy.

Negative pressure wound therapy, also referred to as “vacuum therapy” wound therapy, is an established form of therapy for healing complex wounds. Current forms of negative pressure wound therapy are widely accepted and used broadly to support wound healing. Complex wounds can range from small and acute, to deep and chronic. The current negative pressure wound therapy protocol involves a controlled, sub-atmospheric local wound environment, typically provided by a pump and a sealed wound membrane.

Most current systems in the marketplace use a wound-filler between the wound and wound sealing material. The wound-filler has several characteristics to support wound healing under sub-atmospheric pressure, including providing a consistent distribution of the pressure, distribution and transport of the wound exudates, and protection of the wound.

The two-types of wound-fillers currently in principal use include cotton gauze in the form of a woven mesh material, and foam such as an open cell synthetic foam based on polyurethane or polyvinyl alcohol. Both of these materials present various problems in application, use and removal, including the tendency towards moisture absorption and retention. BRIEF SUMMARY OF THE INVENTION

Therefore, it is an object of the invention to provide an improved negative pressure wound therapy product and method.

It is a further object of the invention to provide a wound-filler with moisture management properties.

It is a further object of the invention to provide an improved negative pressure wound therapy product that is more efficiently manufactured and applied by reason of a lesser number of layers of material being required to achieve the necessary therapeutic results.

To achieve the foregoing and other objects and advantages, a negative pressure wound therapy bandage is provided herein including a three-dimensional knitted fabric formed of fibers that are moisture resistant and that define an exudates transport layer for transporting wound exudates away from a wound.

The bandage may further include a non-adherent material adapted for preventing the exudates transport layer from adhering to the wound during therapy and an antibacterial layer for preventing bacterial growth in and around the wound.

The knitted fabric has an open-mesh knitted construction formed by knitting monofilament yarns, multifilament yarns, or a combination thereof.

The knitted fabric may have a single layer thickness between 2.0 mm and 4.0 mm, and pore size between 1.0 mm and 3.0 mm. The decitex of yarns made from the fibers is preferably between 24 and 75, and yarn types can include nylon, polyester, polypropylene, viscose, polytetrafluoroethylene (PTFE), polyethylene and combinations thereof.

The bandage may further include an adhesive polyurethane film covering.

In another embodiment, a negative pressure wound therapy bandage is provided herein including a polyurethane film backing and a three-dimensional knitted fabric formed of fibers that are moisture resistant and that define an exudates transport layer for transporting wound exudates away from a wound.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed.

### BRIEF DESCRIPTION OF THE DRAWINGS

These and other features, aspects and advantages of the present invention are better understood when the following detailed description of the invention is read with reference to the accompanying drawings, in which:

FIG. 1 is a plan view of a knitted fabric bandage according to a preferred embodiment of the invention, folded into multiple layers for use;

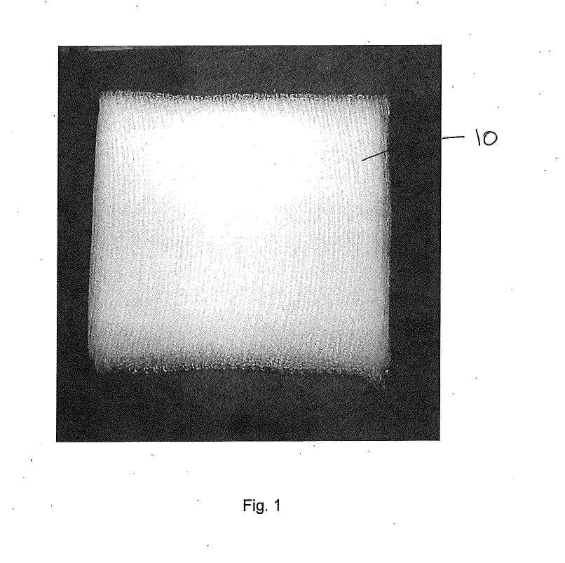

FIG. 2 is a perspective view of a roll form of the knitted fabric bandage according to an embodiment of the invention; and

FIG. 3 is a view showing the knitted fabric bandage and a negative pressure apparatus with which the knitted fabric bandage is used.

### DETAILED DESCRIPTION OF THE INVENTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention. Like reference numbers refer to like elements throughout the various drawings.

Referring to the drawings, an open-knit, water-resistant knitted fabric bandage 10 is used as a wound-filler in a negative pressure wound treatment protocol. The bandage is knitted as opposed to woven, and is a three-dimensional, open-mesh knitted construction formed by knitting monofilament yarns or a combination of monofilament and multifilament yarns into the desired construction. The resulting knitted fabric bandage is therefore easy to cut and to fit to the wound. The resulting knitted fabric bandage is stable under pressure and effectively transports wound exudates away from the wound site. The knitted fabric bandage is easily folded to increase thickness and fill the wound adequately as required for proper treatment.

The wound-filler can be used for both deep wounds and for flat wounds. As described in detail below, the knitted fabric bandage is water-resistant because of both the knitted structure and the yarns from which the fabric is knitted. It is anticipated that the knitted structure may be used as a replacement for several individual layers or in combination with other layers, such as are required in presently available products.

The knitted fabric bandage may include fine filaments, for example >1/24 decitex polyamide both in monofilament and multifilament format.

Another version of the knitted fabric bandage may be formed using polyester monofilament. The thickness of a single layer of the knitted fabric bandage may range from 2.0 mm to 4.0 mm and may have a pore or mesh size from 1.0 mm to 3.0 mm The decitex of the yarns is preferably between 24 and 75, with the yarn types preferably including nylon, polyester, polypropylene, viscose, polytetrafluoroethylene (PTFE), polyethylene and combinations thereof. The combination of yarns may be in either monofilament or multifilament format.

As a result of the inventive knitted construction, the knitted fabric bandage has a very efficient moisture handling capability that reduces or minimizes skin maceration. The open construction of the knitted fabric bandage does not deform under tension, and conforms easily to the wound and anatomy of the patient, thereby offering good comfort characteristics while maintaining and improving the quality of the therapy. The knitted fabric bandage, because of its resiliency characteristics, provides proper cushioning to the wound, while the use of monofilament yarns effectively acts as a moisture transport system, allowing wound exudates to drain away from the wound site. Pressure applied to the wound through the bandage will not close the mesh openings created by the knitted structure, and the knitted fabric bandage is inherently water-resistant.

The knitted fabric bandage may be provided in a three-dimensional, wound-filler knitted structure with a thin multilayer protective cover. The knitted fabric bandage may also be provided in a multilayer wound-filling system with a polyurethane film 12.

A preferred stitch notation for the knitted fabric bandage may be: based on either 4 or 6 knitting bars:
Bar 1, 1-0/2-2/2-3/2-2;
Bar 2, 1-0/1-2/1-0/1-2;
Bar 3, 1-2/1-0/2-2/2-3; and
Bar 4, 2-2/1-0/2-2/2-3;

wherein all bars are fully threaded and every loop consists of 4 ends and Bar 1 and Bar 4 are threaded with 2 ends per guide.

Another fabric construction for the negative pressure wound therapy bandage may be formed on a double needle bar Raschel™ knitting machine according to the following setup:

| | |
|---|---|
| Inlay stitch | 1-0/2-2/2-3/2-2 nylon monofilament |
| Pillar Bar | 1-0/1-3/1-0/1-3 polypropylene multifilament |
| Pillar Bar | 1-3/1-0/1-3/1-0 polypropylene monofilament |
| Inlay Bar | 2-2/1-0/2-2/2-3 nylon monofilament |

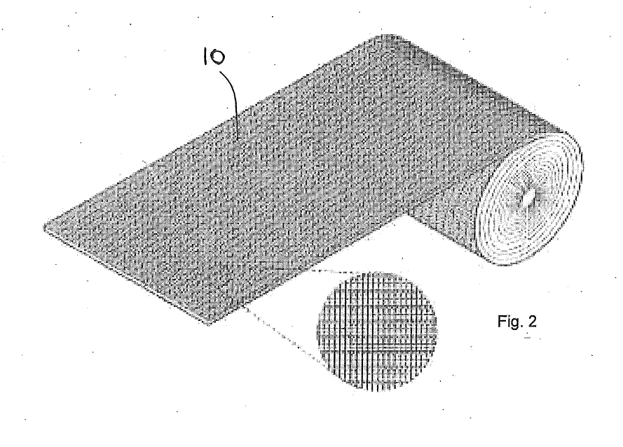

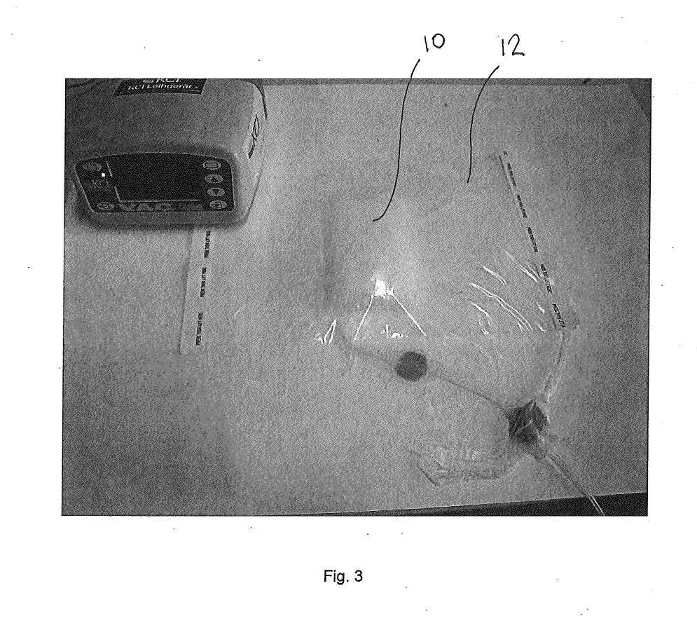

Referring to FIG. 3, a standard dressing set for deep wounds for negative pressure wound therapy may include an adhesive polyurethane film to cover the wound, a tube attachment for the pump, and the wound-filler according to the invention provided in roll form as shown in FIG. 2. A typical deep wound may require a pump with an exudates storage container. A multilayer cover and wound-filler for flat wounds may include the conformable, water-resistant, three-dimensional knitted fabric bandage with or without an additional layer of antibacterial capability. Alternatively, special antibacterial yarns such as anion yarn may be used, for example. The flat wound embodiment is principally intended to transport exudates and secure pressure onto the complete wound. Because this embodiment is designed for smaller wounds, a canister to capture and store exudates may not be necessary in every instance. Instead, a highly-absorbent material may be suitable. One such suitable material is a polyhexamethylene biguanide (PHMB)-impregnated gauze dressing product that limits bacteria growth in and around the wound site. Other anti-bacterial products in the form of solutions are envisioned.

Antibacterial layers such as those discussed above may be used in combination with the inventive knitted bandage, either as a lamination or overlaid without lamination.

The bandage cover is preferably an adhesive polyurethane film used to close the wound. A hydrocolloid version may simplify the application. The resulting advantage is that there is little or no expansion of the hydrocolloid during exudates absorption.

The storage and spreading layer requirements include the necessity of a thin substrate that is able to absorb exudates while exhibiting a low level of expansion and with optimal spreading and transport of exudates. A non-woven material, preferably with highly absorbent characteristics is preferable. One such construction may be approximately 3 mm thick, with a nominal weight of 200 grams per square meter, constructed of approximately 20% superabsorbent fibers with one side laminated with a non-adherent layer. The superabsorbent fibers may be made from the polymerization of acrylic acid blended with sodium hydroxide in the presence of an initiator to form poly-acrylic acid sodium salt, also referred to as sodium polyacrylate. Other materials may be used to form a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

Another construction may be a modified hydro-active material with additional embossed holes, lower absorbency and an added absorbency layer. A hydrogel-dipped nonwoven matrix is a further alternative.

A table of possible structures is set out below:

TABLE 1

| Layer | Dressing 1 | Dressing 2 | Dressing 3 | Dressing 4 |
|---|---|---|---|---|
| Backing | Poly- | Poly- | Poly- | Poly- |
| | urethane | urethane | urethane | urethane |
| | film | film | film | film |
| Core | Non-absorbent | Nonwoven | 3D gel-based | Spacer |
| | hydrogel, | (with or | non-adherent | textile |
| | circular | without | structure | dressing |
| | embossed to | super- | | |
| | support | absorbent | | |
| | exudates | fibers) | | |
| | transport | | | |
| Wound | Not required | Woven | Not required | 3d gel-based |
| Contact | | synthetic | | non-adherent |
| | | mesh | | structure |

The foregoing description provides embodiments of the invention by way of example only. It is envisioned that other embodiments may perform similar functions and/or achieve similar results.

What is claimed is:

1. A negative pressure wound therapy bandage, comprising:
a three-dimensional knitted fabric formed of fibers that are moisture resistant and that define an exudates transport layer for transporting wound exudates away from a wound.

2. The negative pressure wound therapy bandage of claim 1, further comprising a non-adherent material adapted for preventing the exudates transport layer from adhering to the wound during therapy.

3. The negative pressure wound therapy bandage of claim 1, wherein the knitted fabric has an open-mesh knitted construction formed by knitting monofilament yarns, multifilament yarns, or a combination thereof.

4. The negative pressure wound therapy bandage of claim 1, wherein the knitted fabric includes polyamide both in monofilament and multifilament format.

5. The negative pressure wound therapy bandage of claim 1, wherein a thickness of a single layer of the knitted fabric is between 2.0 mm and 4.0 mm, and pore size of the knitted fabric is between 1.0 mm and 3.0 mm.

6. The negative pressure wound therapy bandage of claim 1, wherein the decitex of yarns made from the fibers is preferably between 24 and 75.

7. The negative pressure wound therapy bandage of claim 1, wherein yarn types include nylon, polyester, polypropylene, viscose, polytetrafluoroethylene (PTFE), polyethylene and combinations thereof.

8. The negative pressure wound therapy bandage of claim 1, further comprising an adhesive polyurethane film covering.

9. The negative pressure wound therapy bandage of claim 1, wherein a stitch notation for the knitted fabric is:
based on either 4 or 6 knitting bars:
Bar 1, 1-0/2-2/2-3/2-2;
Bar 2, 1-0/1-2/1-0/1-2;
Bar 3, 1-2/1-0/2-2/2-3; and
Bar 4, 2-2/1-0/2-2/2-3;
wherein all bars are fully threaded and every loop consists of 4 ends and Bar 1 and Bar 4 are threaded with 2 ends per guide.

10. The negative pressure wound therapy bandage of claim 1, wherein the knitted fabric is formed on a double needle bar Raschel™ knitting machine according to the following setup:
Inlay stitch 1-0/2-2/2-3/2-2 nylon monofilament;
Pillar Bar 1-0/1-3/1-0/1-3 polypropylene multifilament;
Pillar Bar 1-3/1-0/1-3/1-0 polypropylene monofilament; and
Inlay Bar 2-2/1-0/2-2/2-3 nylon monofilament.

11. The negative pressure wound therapy bandage of claim 1, further comprising an antibacterial layer in the form of one of a lamination and overlaid without lamination.

12. The negative pressure wound therapy bandage of claim 1, comprising 20% by volume superabsorbent fibers with one side laminated with a non-adherent layer.

13. A negative pressure wound therapy bandage, comprising:
a polyurethane film backing; and
a three-dimensional knitted fabric formed of fibers that are moisture resistant and that define an exudates transport layer for transporting wound exudates away from a wound.

14. The negative pressure wound therapy bandage of claim 13, further comprising a non-adherent material adapted for preventing the exudates transport layer from adhering to the wound during therapy.

15. The negative pressure wound therapy bandage of claim 13, wherein the knitted fabric has an open-mesh knitted construction formed by knitting monofilament yarns, multifilament yarns, or a combination thereof.

16. The negative pressure wound therapy bandage of claim 13, further comprising an antibacterial layer.

17. The negative pressure wound therapy bandage of claim 13, wherein a thickness of a single layer of the knitted fabric is between 2.0 mm and 4.0 mm, and pore size of the knitted fabric is between 1.0 mm and 3.0 mm

18. The negative pressure wound therapy bandage of claim 13, wherein yarn types include nylon, polyester, polypropylene, viscose, polytetrafluoroethylene (PTFE), polyethylene and combinations thereof.

19. The negative pressure wound therapy bandage of claim 13, wherein a stitch notation for the knitted fabric is:
based on either 4 or 6 knitting bars:
Bar 1, 1-0/2-2/2-.3/2-2;
Bar 2, 1-0/1-2/1-0/1-2;
Bar 3, 1-2/1-0/2-2/2-3; and
Bar 4, 2-2/1-0/2-2/2-3;
wherein all bars are fully threaded and every loop consists of 4 ends and Bar 1 and Bar 4 are threaded with 2 ends per guide.

20. The negative pressure wound therapy bandage of claim 13, wherein the knitted fabric is formed on a double needle bar Raschel™ knitting machine according to the following setup:

| | |
|---|---|
| Inlay stitch | 1-0/2-2/2-3/2-2 nylon monofilament; |
| Pillar Bar | 1-0/1-3/1-0/1-3 polypropylene multifilament; |
| Pillar Bar | 1-3/1-0/1-3/1-0 polypropylene monofilament; and |
| Inlay Bar | 2-2/1-0/2-2/2-3 nylon monofilament. |
